(19) United States
(12) Patent Application Publication
(10) Pub. No.: US 2005/0112097 A1
(43) Pub. Date: May 26, 2005

(54) Method for the treatment of cancer

(76) Inventor: Golden S. Hinton, Athens, GA (US)

Correspondence Address: TECHNOPROP COLTON, L.L.C., P O BOX 567685, ATLANTA, GA, 311567685, US

(21) Appl. No.: 10/723,272

(22) Filed: Nov. 26, 2003

(51) Int. Cl. A61K 48/00

(52) U.S. Cl. 424/93.2; 435/454

(57) ABSTRACT

A method of treating and eliminating cancer within a patient by repairing the broken DNA that causes normal cells to become cancerous. Then DNA and/or RNA from bacteria are introduced into the patient's bloodstream to produce septicemia, which then is countered by the appropriate antibiotics. The bacterial cells rupture with spillage of the cell's content. The DNA and/or RNA spilled is used by repair enzymes to physically repair the cancer cell's DNA, thus eliminating the cancer. Any microorganism containing in its genome RNA, DNA, or both, live or attenuated, including any and all means by which they may be introduced by devised or natural means, which may or may not require an antibiotic are variations upon the theme of this method.

### BACKGROUND OF THE INVENTION

1. Technical Field

This method relates to the physical replacement of broken or damaged cancer cell deoxyribonucleic acid (DNA) by either indigenous or acquired repair enzymes. This method in “spontaneous remission” has resulted in the cure for even far advanced cancer.

2. Prior Art

The treatment of cancer includes that of surgery, chemotherapy, and of radiation. Of late, the use of gene therapy that relies upon a gene's ability to produce a key protein where and when needed is being attempted. Our method differs markedly from these and from all other therapies as will be seen.

As discussed in Chromosomal Fragility Can Unstable Segments Of DNA Explain Some Cancers, by Nathan Seppa, Science News, Volume 154, Page 3 (14 Nov. 1998) (referred to as the Seppa Article), broken (undamaged) DNA of cells that are cancerous can be repaired by physically replacing broken DNA with portions of DNA from bacteria or other sources in the presence of repair enzymes. This theory is reiterated in Gene Therapy: Safer And Virus-Free?, Science, Volume 294, Page 1638 (23 Nov. 2001) (referred to as the Science News Focus Article). However, these two articles do not put forth a method for replacing or repairing DNA or RNA as disclosed herein.

Spontaneous remission of cancer also is known. An illustrative case of spontaneous remission is reported in It's Just A Miracle, by Virginia Anderson, Atlanta Journal-Constitution, 13 Nov. 2003. However, one cannot count on fortuitous spontaneous remission, and it would be helpful to be able to induce or create spontaneous remission.

It is widely known that a large number of those with cancer die each year from cancer. It is also true that billions of dollars are spent yearly for cancer treatment and on cancer research, yet it remains a very much unsolved problem. Thus, there exists a need for a successful and more economical method for the treatment of cancer. The present invention is directed to this need and others related to the treatment and cure of cancer.

### BRIEF SUMMARY OF THE INVENTION

The present method introduces live bacteria into a cancer patient by intravenous infusion to produce septicemia. Antibiotics then are given to kill these bacteria. With the rupture of their cellular membranes, DNA and ribonucleic acid (RNA) (and other cellular components) are spilled into the patient's bloodstream. Bits and particles of molecular DNA and RNA become available for the patient's repair enzymes to use to repair the damage and breakage that give rise to cancerous cells. With this repair, the cancer is eliminated.

Any microorganism containing in its genome RNA, DNA, or both, live or attenuated, including any and all means by which they may be introduced by devised or natural means, which may or may not require an antibiotic are variations upon the theme of this method.

### DETAILED DESCRIPTION OF THE PREFERRED EMBODIMENTS

The accessibility of cancer involving the skin makes discovery and treatment easier, while that internal to the skin is the reverse. It is cancer internal to the skin to which this method primarily is aimed.

The most satisfactory of cancer cures internally is the phenomenon known as “spontaneous remission” or “spontaneous regression”, whereupon virulent cancers of many types simply disappear seemingly without reason. It is research into this subject that has led to a new therapy and this new method for the treatment of cancer.

A heretofore unpublished and undisclosed report, included below under the “Case Study” section, provides the basis for the present invention. This report, along with the Seppa Article and the Science News Focus Article both of which are incorporated into this disclosure and specification by this reference, suggests and we theorize that broken (undamaged) DNA of cells that are cancerous can be repaired by physically replacing broken DNA with portions of DNA from bacteria or other sources in the presence of repair enzymes.

As used in this specification and claims, the term DNA/RNA includes both DNA and RNA, either separately or together, and all of their component and constituent parts, as well as their structures. Further, any microorganism containing in its genome RNA, DNA, or both, live or attenuated, including any and all means by which they may be introduced by devised or natural means, which may or may not require an antibiotic are alternative embodiments of this method.

The treatment method is to introduce live bacteria into the patient's bloodstream. This produces a septicemia (infection) with fever and signs and symptoms consistent with infection. Sensitivity tests are performed to determine the most lethal antibiotics to the bacteria chosen to be infused. Once septicemia is noted to be present, the fever produced is allowed to continue for 24 to 48 hours depending upon the degree of severity. The appropriate antibiotics then are given to the patient and once their effectiveness is realized by a return to a normal temperature and a symptom free patient, the patient is discharged from the hospital. Follow up in the physician's office is performed for both cancer and the induced infection.

We theorize that cancer is the result of broken or otherwise damaged DNA structure of a cell, causing the cell to become cancerous. Though a patient's immune system often can kill the bacteria of infection, the present method relies upon the much greater certainty of sensitivity test antibiotics to kill the bacteria and that cause the rupture of the bacterial cell membrane with spillage of DNA, RNA, and the other intracellular components of the cell into the patient's bloodstream. Repair enzymes then use the molecular bits and pieces of DNA and RNA to repair the DNA structure of the cancerous cell and eliminates the cancer. Other cellular components, other than DNA or RNA, may or may not contribute to the elimination of cancer.

We choose to introduce the bacteria into the patient by intravenous infusion of a nontoxic solution that contains the chosen bacteria. These bacteria also may be introduced by injection by the respiration of surrounding air, by nasal spray, or by other means. Other microorganisms that contain DNA or RNA also may be used to provide the needed components from DNA or RNA.

The advantage of this method is to increase the probability of eliminating cancer from the patient, to reduce the monetary cost of treatment as well as the discomfort, the time expended, and the side effects associated with contemporary treatment.

### Case Study

A relatively recent single case study and a review of other cases of spontaneous remission, sometimes referred to as spontaneous regression, reported in the literature of the early and middle decades of the 20th century lead to the proposal of a theory to explain the cause of cancer.

The theory is based on observations made after the review of the case histories of patients that had spontaneous regression of their cancer. This theory suggests that broken (damaged) DNA of cells that are cancerous can be repaired by physically replacing broken DNA with undamaged DNA from bacteria or other sources in the presence of repair enzymes. This method of treatment may induce permanent cure of cancer in otherwise fatal cases.

In March or 1997, G. B. was in my (the inventor) ophthalmology office for an eye examination. A routine history of her health status since I had last seen her in August 1994 prompted the reply: “I was supposed to die but I didn't.” She related that she had an advanced case of multiple myeloma treated with chemotherapy by a local oncologist, but had been dismissed from his care upon his having no further treatment to offer her. She was therefore prepared for death, but in the ensuing months, felt better and consulted an oncologist in another town who told her that she had no evidence of myeloma. She reported a hospitalization for fever of unknown origin in October of 1995. This preceded her dismissal from further oncology treatment by several weeks.

A phone call to her local treating oncologist confirmed the accuracy of her story to me. He was unaware of her spontaneous remission and that she continued to live.

A review of her hospital record of October/November 1995 showed that a blood culture had grown salmonella type D bacteria. This bacterium explained her fever and she responded to antibiotics given her by an infectious disease consultant by becoming afebrile. A week or so after her dismissal from the hospital, she was seen by her oncologist who, as related-earlier, was unable to offer further treatment options.

A compendium of cases of spontaneous remission from the early and middle decades of the 20^{th} century entitled Spontaneous Remission by Brendan O'Rogan and Caryle Hirchberg and sponsored by the Institute of Noetic Science was reviewed. A noteworthy finding was the presence of infections of varying types with fever in a number of these reports. This was also noted by treating physicians, but attempts to apply this association in the treatment of other cancer patients was abandoned for unknown reasons.

Today it is known that there are “fragile sites, chromosomal regions that are prone to break apart or suffer mutations” and that “there is growing evidence of breaks at these fragile locations in cancer cells”. Seppa Article. It is also known that “naked DNA injections are the simplest nonviral gene delivery method”. Science News Focus Article.

The information above leads to the conclusion that G. B.'s myeloma and other cases of cancer with infections and fever, acquired their remission/regression of cancer via their infections. G. B.'s bacteremia was eliminated by antibiotics that acted to rupture the cytoplasmic membranes of the salmonella. This rupture released the bacteria's “naked” DNA into the bloodstream. These DNA particles then provided the raw materials for repair enzymes to anneal or otherwise repair the broken DNA sites. Breakage or weakness of the structure of cells (whether inherited or acquired) that become cancerous causes the cell to replicate without end in a dysfunctional manner. The host organism is damaged to the point that death of the organism eventually occurs.

The above detailed description of the preferred embodiments and case study are for illustrative purposes only and are not intended to limit the scope and spirit of the invention, and its equivalents, as defined by the appended claims. One skilled in the art will recognize that many variations can be made to the invention disclosed in this specification without departing from the scope and spirit of the invention.

1. A method of the introduction of normal DNA within bacteria that are killed by antibiotics spill DNA/RNA into the bloodstream which DNA/RNA supplies material for the repair for broken cancer cell DNA.

2. The method as claimed in claim 1, including the spillage of other components of the bacteria which may contribute a therapeutic effect which is now not known.

3. The method as claimed in claim 1, including any microorganisms that contain DNA/RNA, whether live or attenuated.

4. The method as claimed in claim 1, using nontoxic intravenous infusion to introduce bacteria or other microorganisms into the bloodstream.

5. The method as claimed in claim 4, including all other means for introducing bacteria and any other microorganisms as by injection, inspiration of organisms from surrounding air, by nasal spray or any other devised or natural means.

6. The method as claimed in claim 1, whether rupture of cells is accomplished by antibiotics, the immune system, or any other mechanism of rupture of a cell.

7. The method as claimed in claim 1, including unenclosed (naked) DNA/RNA as well as those enclosed by a cellular membrane or an artificial enclosure.

8. A method for the treatment of cancerous cells in a patient comprising the steps of:
a) introducing a sufficient quantity of bacteria into the patient's bloodstream to cause septicemia in the patient;
b) allowing the septicemia to proceed for at least 24 hours; and
c) treating the septicemia with a medicinally effective quantity of an antibiotic appropriate for the selected bacteria so as to control the septicemia, wherein at least a portion of the bacteria cells rupture and release intracellular components including the bacteria's DNA/RNA into the patient's bloodstream and whereby the patient's repair enzymes use the bacteria's DNA/RNA to repair damaged DNA/RNA in at least a portion of the cancerous cells.

9. The method as claimed in claim 8, wherein the bacteria are introduced into the patient's bloodstream by a method selected from the group consisting of intravenous infusion, injection, inspiration, respiration, nasal spray, subcutaneous, intra-muscular, and intra-abdominal.

10. The method as claimed in claim 8, wherein the intracellular components including the bacteria's DNA/RNA include portions of the bacteria's DNA/RNA.

11. The method as claimed in claim 8, wherein the septicemia is allowed to proceed for between 24 and 48 hours.

12. A method for the treatment of cells containing damaged DNA in a patient comprising the steps of:
a) introducing a sufficient quantity of a DNA/RNA-containing microorganism into the patient's bloodstream to cause septicemia in the patient;
b) allowing the septicemia to proceed for at least 24 hours; and
c) treating the septicemia with a medicinally effective quantity of an antibiotic appropriate for the selected DNA/RNA-containing microorganism so as to control the septicemia, wherein at least a portion of the DNA/RNA-containing microorganism cells rupture and release intracellular components including the DNA/RNA-containing microorganism's DNA/RNA into the patient's bloodstream and whereby the patient's repair enzymes use the DNA/RNA-containing microorganism's DNA/RNA to repair damaged DNA in at least a portion of the cells containing damaged DNA.

13. The method as claimed in claim 12, wherein the DNA/RNA-containing microorganism is introduced into the patient's bloodstream by a method selected from the group consisting of intravenous infusion, injection, inspiration, respiration, nasal spray, subcutaneous, intramuscular, and intra-abdominal.

14. The method as claimed in claim 12, wherein the intracellular components including the DNA/RNA-containing microorganism's DNA/RNA include portions of the DNA/RNA-containing microorganism's DNA/RNA.

15. The method as claimed in claim 12, wherein the septicemia is allowed to proceed for between 24 and 48 hours.

16. The method as claimed in claim 12, wherein the DNA/RNA-containing microorganism is a bacteria.

17. The method as claimed in claim 12, wherein the cells containing damaged DNA are cancerous cells.

18. A method for the treatment of cells containing damaged DNA in a patient comprising the steps of:
a) introducing a sufficient quantity of a DNA/RNA-containing microorganism into the patient's bloodstream to cause septicemia in the patient, wherein the DNA/RNA-containing microorganism is introduced into the patient's bloodstream by a method selected from the group consisting of intravenous infusion, injection, inspiration, respiration, nasal spray, subcutaneous, intramuscular, and intra-abdominal;
b) allowing the septicemia to proceed for a period of between 24 and 48 hours; and
c) treating the septicemia with a medicinally effective quantity of an antibiotic appropriate for the selected DNA/RNA-containing microorganism so as to control the septicemia, wherein at least a portion of the DNA/RNA-containing microorganism cells rupture and release intracellular components including complete and partial portions of the DNA/RNA-containing microorganism's DNA/RNA into the patient's bloodstream and whereby the patient's repair enzymes use the DNA/RNA-containing microorganism's DNA/RNA to repair damaged DNA in at least a portion of the cells containing damaged DNA.

19. The method as claimed in claim 18, wherein the DNA/RNA-containing microorganism is a bacteria.

20. The method as claimed in claim 18, wherein the cells containing damaged DNA are cancerous cells.

21. The method as claimed in claim 19, wherein the cells containing damaged DNA are cancerous cells.
